(19) United States
(12) Patent Application Publication
(10) Pub. No.: US 2004/0248863 A1
(43) Pub. Date: Dec. 9, 2004

(54) Combination of a ppar-alpha ligand and glucocorticoid for the treatment or prevention of inflamation

(76) Inventor: Samuel D. Wright, Westfield, NJ (US)

Correspondence Address: MERCK AND CO INC, P O BOX 2000, RAHWAY, NJ, 070650907

(21) Appl. No.: 10/481,869

(22) PCT Filed: Jun. 19, 2002
(86) PCT No.: PCT/US02/19520

Related U.S. Application Data
(60) Provisional application No. 60/300,263, filed on Jun. 22, 2001.

(51) Int. Cl. A61K 31/573

(52) U.S. Cl. 514/171

(57) ABSTRACT

The present invention relates to a method for treating or preventing an inflammatory disease or condition in a patient in need thereof comprising concomitantly administering to said patient a PPARα agonist and a glucocorticoid in amounts that are effective to treat or prevent the inflammatory disease or condition. Pharmaceutical compositions for treating or preventing an inflammatory disease or condition are also encompassed.

### BACKGROUND OF THE INVENTION

A key step in the process of inflammation is the ingress of leukocytes into inflammed tissue. During this process, the affected tissue responds to the inflammatory insult by the elaboration of cytokines, chemotactic factors, and adhesion molecules. These molecules serve in the attraction of leukocytes. Simultaneously, leukocytes respond to these factors by adhesion, migration, and elaboration of further cytokines and chemotactic factors. It is thus clear that inflammation requires the response of both the target tissue and the leukocytes. This dual contribution is illustrated by the failure of inflammation to occur in animals deficient in leukocyte adhesion molecules (beta-2 integrins) or in animals deficient in the tissue expression of the counterreceptor for integrins, ICAM-1. This invention involves administration of an agent that binds to transcription factors both in tissues and in leukocytes.

Peroxisome proliferator-activated receptors (PPARs) are transcription factors belonging to the nuclear receptor supergene family. Three distinct PPARs, termed α, δ and γ, have been described. Each one is encoded by a separate gene. PPARs are characterized by distinct tissue distribution patterns and metabolic functions.

PPARγ is a transcription factor expressed in adipose tissues, cells of the colon, and in tissue macrophages. Several studies indicate that ligands for PPARγ suppress the expression of proinflammatory molecules (Nature 391:82, Nature 391;79: Cell 93, 241; Cell 93; 229 (1998)) and may have anti-inflammatory action in vivo (JCI 104: 383, 1999). It is noteworthy that strong anti-inflammatory activity was observed in a model of colon inflammation and PPARγ is strongly expressed in the colon. PPARα is a homologous transcription factor with a distinct expression pattern being present in liver, monocytes, smooth muscle cells and other tissues. Recent studies indicate that agonists of PPARα blunt production of pro-inflammatory cytokines (Nature 393: 790 (1998), Circulation 99: 3125 (1999)).

### SUMMARY OF THE INVENTION

The present invention relates to a method for treating or preventing an inflammatory disease or condition in a patient in need thereof comprising concomitantly administering to said patient a PPARα agonist and a glucocorticoid in amounts that are effective to treat or prevent the inflammatory disease or condition. Pharmaceutical compositions for treating or preventing an inflammatory disease or condition are also encompassed.

### DETAILED DESCRIPTION OF THE INVENTION

The present invention relates to a method for treating or preventing an inflammatory disease or condition in a patient in need thereof comprising concomitantly administering to said patient a PPARα agonist and a glucocorticoid in amounts that are effective to treat or prevent the inflammatory disease or condition.

An embodiment of the invention encompasses a method for treating an inflammatory disease or condition in a patient in need thereof comprising concomitantly administering to said patient a PPARα agonist and a glucocorticoid in amounts that are effective to treat the inflammatory disease or condition.

Another embodiment of the invention encompasses a method for preventing an inflammatory disease or condition in a patient in need thereof comprising concomitantly administering to said patient a PPARα agonist and a glucocorticoid in amounts that are effective to prevent the inflammatory disease or condition.

A preferred glucocorticoid for the methods of the present invention is dexamethasone. Other glucocorticoids include, for example, aldosterone, beclomethasone, betamethasone, budesonide, cloprednol, cortisone, cortivazol, eoxycortone, desonide, desoximetasone, difluorocortolone, luclorolone, flumethasone, flunisolide, fluocinolone, luocinonide, fluocortin butyl, fluorocortisone, fluorocortolone, fluorometholone, flurandrenolone, fluticasone, alcinonide, hydrocortisone, comethasone, meprednisone, methylprednisolone, mometasone, paramethasone, prednisolone, prednisone, tixocortol, triamcinolone, and others, and their respective pharmaceutically acceptable derivatives, such as beclomethasone diproprionate, dexamethasone 21-isonicotinate, fluticasone propionate, icomethasone enbutate, tixocortol 21-pivalate, triamcinolone acetonide, and others.

Another embodiment of the invention encompasses a method for treating or preventing an inflammatory disease or condition in a patient in need thereof comprising administering to said patient a PPARα agonist in an amount that is effective to treat or prevent the inflammatory disease or condition.

An embodiment of the invention encompasses a method for treating an inflammatory disease or condition in a patient in need thereof comprising administering to said patient a PPARα agonist in an amount that is effective to treat of the inflammatory disease or condition.

Another embodiment of the invention encompasses a method for preventing an inflammatory disease or condition in a patient in need thereof comprising administering to said patient a PPARα agonist in an amount that is effective to prevent the inflammatory disease or condition.

For purposes of this specification, concomitant means that the two drugs are administered either in combination or that one drug is administered separately while the first drug is present in a therapeutically effective amount.

Another embodiment of the invention encompasses a method for treating or preventing an inflammatory disease or condition in a patient in need thereof comprising concomitantly administering to said patient a PPARα agonist and a glucocorticoid in amounts that are effective to treat or prevent the inflammatory disease or condition, wherein the inflammatory disease or condition is inflammatory bowel syndrome.

Another embodiment of the invention encompasses a method for treating or preventing an inflammatory disease or condition in a patient in need thereof comprising concomitantly administering to said patient a PPARα agonist and a glucocorticoid in amounts that are effective to treat or prevent the inflammatory disease or condition, wherein the inflammatory disease or condition is arthritis. Within this group is encompassed the above method wherein the inflammatory disease or condition is selected from the group consisting of: rheumatoid arthritis, ankylosing spondylitis, gout, psoriasis, osteoarthritis, and juvenile arthritis.

The invention also encompasses a pharmaceutical composition comprising a PPARα agonist with a glucocorticoid in combination with a pharmaceutically acceptable carrier.

Compounds that are PPARα agonists are known in the art and include fenofibrate, clofibrate, gemfibrozil and benzafibrate. This invention does not encompass dual PPARα/γ agonists. Other examples of compounds which are PPARα agonists are found in the following patents and published applications: WO 97/28115 published on Aug. 7, 1997; WO 00/78312 published on Dec. 28, 2000; WO 00/78313 published on Dec. 28, 2000; U.S. Pat. No. 5,847,008 granted on Dec. 8, 1998; U.S. Pat. No. 5,859,051 granted on Jan. 12, 1999; U.S. Pat. No. 6,008,237 granted on Dec. 28, 1999; U.S. Pat. No. 6,090,836 granted on Jul. 18, 2000; U.S. Pat. No. 6,090,839 granted on Jul. 18, 2000; U.S. Pat. No. 6,160,000 granted on Dec. 12, 2000; and U.S. Pat. No. 6,200,998 granted on Mar. 13, 2001, all of which are hereby incorporated by reference in their entirety.

Utilities

Compounds that are PPARα agonists are useful for the treatment or prevention of inflammatory diseases or conditions. For example, the present invention encompasses the treatment or prevention of arthritis, including but not limited to rheumatoid arthritis, ankylosing spondylitis, gout, psoriasis, osteoarthritis, systemic lupus erythematosus and juvenile arthritis. The invention also includes the treatment of: asthma, bronchitis, menstrual cramps, tendinitis, bursitis, and skin related conditions such as psoriasis, eczema, burns and dermatitis; gastrointestinal conditions such as inflammatory bowel disease, Crohn's disease, gastritis, irritable bowel syndrome and ulcerative colitis; inflammation in such diseases as vascular diseases, migraine headaches, periarteritis nodosa, thyroiditis, aplastic anemia, Hodgkin's disease, sclerodoma, rheumatic fever, vasculitis, systemic lupus erythematosis (SLE), Alzheimer's disease, atherosclerosis, acute respiratory distress syndrome (ARDS), myasthenia gravis, multiple sclerosis, sarcoidosis, nephrotic syndrome, Behcet's syndrome, polymyositis, gingivitis, hypersensitivity, swelling occurring after injury, and myocardial ischemia; ophthalmic diseases, such as retinitis, retinopathies, conjunctivitis, uveitis, ocular photophobia, and of acute injury to the eye tissue; and the treatment of pulmonary inflammation, such as that associated with viral infections and cystic fibrosis.

Pharmaceutical Compositions

The pharmaceutical compositions of the present invention comprise a PPARα agonist as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the present compounds can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form, in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The present compounds may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethypiperideine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to PPARα agonists or compounds which are PPARα agonists include the pharmaceutically acceptable salts thereof.

Optical Isomers—Diastereomers—Geometric Isomers—Tautomers

The compounds of the present invention may contain one or more asymmetric centers and can thus occur as racemates and racemic mixtures, single enantiomers, diastereomeric mixtures and individual diastereomers. The present invention is meant to comprehend all such isomeric forms.

The compounds encompassed by the present invention may contain olefinic double bonds, and unless specified otherwise, are meant to include both E and Z geometric isomers.

The compounds encompassed by the present invention may exist with different points of attachment of hydrogen, referred to as tautomers. Such an example may be a ketone and its enol form, known as keto-enol tautomers. The individual tautomers as well as mixtures thereof are encompassed with compounds of Formula II and IIa.

The compounds encompassed by the present invention may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of the compounds of the present invention may be obtained by stereospecific synthesis using optically pure starting materials or reagents of known configuration.

Administration and Dose Ranges

Any suitable route of administration may be employed for providing a mammal, and especially a human, with an effective dosage of the present combination therapy for the treatment or prevention of an inflammatory disease or condition. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably the compounds are administered orally.

The effective dosages of the active ingredients employed may vary depending on the particular compound employed, the mode of administration, the condition being treated and the severity of the condition being treated. Such dosage may be ascertained readily by a person skilled in the art.

When treating or preventing an inflammatory disease or condition generally satisfactory results are obtained when the PPARα agonist is administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

Effective dosages of glucocorticoids are well known in the art. For example, dexamethasone tablets are available at potencies of 0.5 mg, 0.75 mg and 4 mg.

When administered concomitantly, either a single or as a separate pharmaceutical composition for the treatment or prevention of an inflammatory disease or condition, the PPARα agonist and glucocorticoid are presented in a ratio that is consistent with the manifestation of the desired effect. In particular, the ratio by weight of the PPARα agonist to the glucocorticoid will suitably be approximately between 0.001 to 1 and 1000 to 1, and especially between 0.01 to 1 and 100 to 1.

Combination Therapy

The compounds of the present invention for use in treating or preventing an inflammatory disease or condition may be used in combination with other drugs for the treatment or prevention of the inflammatory disease or condition. Such other drugs may be administered, by a route and in an amount commonly used therefor, contemporaneously or sequentially with a compound of the present invention. When a compounds of the present invention are used contemporaneously with one or more other drugs, a pharmaceutical composition in unit dosage form containing such other drugs and the PPARα agonist in combination with a glucocorticoid or alone is preferred. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compounds of the present invention.

Biological Assays

Standardized Cell-Based GAL4 Chimeric Receptor Transactivation Assay (Cell-Based Transactivation Assay)

The following assay is also described in: Berger J, Leibowitz M D, Doebber T W, Elbrecht A, Zhang B, Zhou G, Biswas C, Cullinan C A, Hayes N S, Li Y, Tanen M, Ventre J, Wu M S, Berger G D, Mosley R, Marquis R, Santini C, Sahoo S P, Tolman R L, Smith R G, Moller D E. Novel peroxisome proliferator-activated receptorγ (PPARγ) and PPARδ ligands produce distinct biological effects, 1999 J Biol Chem 274: 6718-6725, herein incorporated by reference in its entirety:

Expression constructs are prepared by inserting cDNA sequences encoding the ligand binding domains of human PPARγ or PPARα adjacent to the yeast GAL4 transcription factor DNA binding domain in the mammalian expression vector pcDNA3 to create pcDNA3-hPPARγ/QAL4 and pcDNA3-hPPARα/GAL4, respectively. The GAL4-responsive reporter construct, pUAS(5X)-tk-luc, contains 5 copies of the GAL4 response element placed adjacent to the thymidine kinase minimal promoter and the luciferase reporter gene. The transfection control vector, pCMV-lacZ, contains the galactosidase Z gene under the regulation of the cytomegalovirus promoter. COS-1 cells are seeded at 1.2×10⁴ cells/well in 96 well plates in Dulbecco's modified Eagle medium (high glucose) containing 10% charcoal stripped fetal calf serum, nonessential amino acids, 100 units/ml Penicillin G and 100 μg/ml Streptomycin sulfate at 37° C. in a humidified atmosphere of 10% CO₂. After 24 h, transfections are performed with Lipofectamine (Gibco-BRL, Gaithersburg, Md.) according to the instructions of the manufacturer. Transfection mixes contain 0.00075 μg of PPARγ/GAL4 or PPARα/GAL4 expression vector, 0.045 μg of reporter vector pUAS(5X)-tk-luc and 0.0002 μg of pCMV-lacZ vector as an internal control of transfection efficiency. Compounds are characterized by incubation with transfected cells for 48 h across a range of 8-12 concentrations from 0.1 nM to 50 μM. Cell lysates are prepared from washed cells using Reporter Lysis Buffer (Promega) according to the manufacturer's directions. Luciferase activity in cell extracts is determined using Luciferase Assay Buffer (Promega) in a ML3000 luminometer (Dynatech Laboratories). β-galactosidase activity is determined using β-D-galactopyranoside (Calbiochem-Novabiochem, LaJolla, Calif.) as described by Hollons and Yoshimura (Anal. Biochem, 182,411-418, 1989). Rosiglitazone can be used as a standard for human PPARγ activity. EC₅₀ values for Rosiglitazone in the hPPARγ/GAL4 assay usually range from 2040 nM. Fenofibrate can be used as a standard for hPPARα activity. EC₅₀ values for Fenofibrate in the hPPARα/GAL4 assay usually range from 5-20 μM. Similarly, methods involving the co-transfection of full-length PPARγ or PPARγ along with a relevant reporter gene into one of several mammalian (or yeast) cell types could be employed as an alternative method to identify compounds with both PPARα and PPARγ agonist activity.

Cell-Free Co-Activator Association Assay

This assay measures the ability of compounds to promote the association of PPARγ (or its isolated ligand binding domain) or PPARα (or its isolated ligand binding domain) with a protein (or portion of a protein) that is (or is derived from) a co-activator molecule such as Creb Binding Protein (CBP) or Steroid Receptor Coactivator 1 (SRC-1) and can be used to identify compounds with both PPARα and PPARγ agonist activity. This assay is described in: Zhou G, Cummings R, Li Y, Mitra S, Wilkinson H, Elbrecht A, Hermes J D, Schaeffer J M, Smith R G, Moller D E. Nuclear receptors have distinct affinities for co-activators: characterization by fluorescence resonance energy transfer. Mol Endocrinol 1998 12:1594-1604, herein incorporated by reference in its entirety.

Human PPARα and PPARγ binding assays

An alternative to measuring agonist activity of compounds in cell-based transactivation assays or cell-free co-activator association assays is to determine that compounds can function as ligands by binding to both PPARγ and PPARα. Compounds with half-maximal concentration potencies (IC₅₀'s or KI's) for displacement of radioligand binding to hPPARγ vs. hPPARα that differ by less than 30-fold and preferably less than 10-fold can be considered as dual ligands. For these assays, the methods described below can be employed (as also described in: Berger J, Leibowitz M D, Doebber T W, Elbrecht A, Zhang B, Zhou G, Biswas C, Cullinan C A, Hayes N S, Li Y, Tanen M, Ventre J, Wu M S, Berger G D, Mosley R, Marquis R, Santini C, Sahoo S P, Tolman R L, Smith R G, Moller D E. Novel peroxisome proliferator-activated receptorγ (PPARγ) and PPARδ ligands produce distinct biological effects, 1999 J Biol Chem 274: 6718-6725, herein incorporated by reference in its entirety):

Human PPARγ₂ and human PPARα were expressed as a GST-fusion protein in E. coli. The full length human cDNA for PPARγ₂ was subcloned into the pGEX-2T expression vector (Pharmacia). The full length human cDNA for PPARα was subcloned into the pGEX-KT expression vector (Pharmacia). E. coli containing the respective plasmids were propagated, induced, and harvested by centrifugation. The resuspended pellet was broken in a French press and debris was removed by centrifugation at 12,000×g. Recombinant human PPAR receptors were purified by affinity chromatography on glutathione sepharose. After application to the column, and one wash, receptor was eluted with glutathione. Glycerol (10%) was added to stabilize the receptor and aliquots were stored at −80° C.

For each assay, an aliquot of receptor was incubated in TEGM (10 mM Tris, pH 7.2, 1 mM EDTA, 10% glycerol, 7 μL/100 ml β-mercaptoethanol, 10 mM Na molybdate, 1 mM dithiothreitol, 5 μg/mL aprotinin, 2 μg/mL leupeptin, 2 μg/mL benzamidine and 0.5 mM PMSF) containing 0.1% non-fat dry milk and 10 nM [³H₂] L-746,962, (21 Ci/mmole), ±test compound. Assays were incubated for ˜16 hr at 4° C. in a final volume of 150 μL. Unbound ligand was removed by incubation with 100 μL dextran/gelatin-coated charcoal, on ice, for 10 min. After centrifugation at 3000 rpm for 10 min at 4° C., 50 μL of the supernatant fraction was counted in a Topcount. In this assay the K_{D} for L-746,962 is ≈1 nM.

For a human PPARα binding assay, an aliquot of receptor was incubated in TEGM (10 mM Tris, pH 7.2, 1 mM EDTA, 10% glycerol, 7 μL/100 ml β-mercaptoethanol, 10 mM Na molybdate, 1 mM dithiothreitol, 5 μg/mL aprotinin, 2 μg/mL leupeptin, 2 μg/mL benzamide and 0.5 mM PMSF) containing 0.1% non-fat dry milk and 5.0 nM [³H₂]L-783483, ±test compound. Assays were incubated for ˜16 hr at 4° C. in a final volume of 150 μL. Unbound ligand was removed by incubation with 100 μL dextran/gelatin-coated charcoal, on ice, for ˜10 min. After centrifugation at 3000 rpm for 10 min at 4° C., 50 μL of the supernatant fraction was counted in a Topcount.

Cell Proliferation Assay

This assay measures the ability of cells to convert MTS tetrazolium into formazan, using the AQᵤₑₒᵤₛ cell proliferation assay kit (Promega, Madison, Wis.). This conversion is presumably accomplished by NADPH or NADH produced by dehydrogenase enzymes in metabolically active cells. The assay is described in Shu, et al., Biochemical and Biophysical Research Communications, vol. 267, pp. 345-349 (2000).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method for treating or preventing an inflammatory disease or condition in a patient in need thereof comprising concomitantly administering to said patient a PPARα agonist and a glucocorticoid in amounts that are effective to treat or prevent the inflammatory disease or condition.

2. A method for treating an inflammatory disease or condition in a patient in need thereof comprising concomitantly administering to said patient a PPARα agonist and a glucocorticoid in amounts that are effective to treat the inflammatory disease or condition, in accordance with claim 1.

3. A method for preventing an inflammatory disease or condition in a patient in need thereof comprising concomitantly administering to said patient a PPARα agonist and a glucocorticoid in amounts that are effective to prevent the inflammatory disease or condition, in accordance with claim 1.

4. The method according to claim 1 wherein the glucocorticoid is dexamethasone.

5. The method according to claim 1 wherein the inflammatory disease or condition is inflammatory bowel syndrome.

6. The method according to claim 1 wherein the inflammatory disease or condition is arthritis.

7. The method according to claim 1 wherein the inflammatory disease or condition is rheumatoid arthritis.

8. The method according to claim 1 wherein the inflammatory disease or condition is ankylosing spondylitis.

9. The method according to claim 1 wherein the inflammatory disease or condition is gout.

10. The method according to claim 1 wherein the inflammatory disease or condition is psoriasis.

11. The method according to claim 1 wherein the inflammatory disease or condition is osteoarthritis.

12. The method according to claim 1 wherein the inflammatory disease or condition is juvenile arthritis.

13. A pharmaceutical composition comprising a PPARα agonist and a glucocorticoid in combination with a pharmaceutically acceptable carrier.

14. Cancelled.
